(19) United States
(12) Patent Application Publication
(10) Pub. No.: US 2025/0348719 A1
(43) Pub. Date: Nov. 13, 2025

(54) METHOD FOR PERSONAL NEUROMORPHIC EMULATION

(71) Applicant: Brain Electrophysiology Laboratory Company, LLC, Eugene, OR (US)

(72) Inventors: Don M. Tucker, Eugene, OR (US); Phan Luu, Eugene, OR (US)

(21) Appl. No.: 19/072,686

(22) Filed: Mar. 6, 2025

(51) Int. Cl. G06N 3/06; A61B 5/00; A61B 5/369

(52) U.S. Cl. CPC G06N 3/061; A61B 5/369; A61B 5/4806

(57) ABSTRACT

A method for personal neuromorphic emulation. The method may include constructing a computerized anatomical network model of neurons in the brain, the anatomical network model defining adjustably weighted connections between the modeled neurons and producing EEG signals as outputs; taking first hdEEG data from the subject while the subject's brain is awake and performing one or more first intellectual tasks; and adjusting the weights of the weighted connections of the anatomical network model so as to drive the EEG signal outputs of the anatomical network model toward the first hdEEG data.

### FIELD OF THE INVENTION

This invention is in the field of computational emulation of the human mind in a durable informatic appliance. Progress in several related scientific fields has progressed to the point that it is now feasible to design distributed neuronal models of cognitive and emotional function constructed with sufficient fidelity to an individual brain to replicate an individual's personality and subjective experience in digital (or other informatic) form. These fields include artificial intelligence, developmental neuropsychology, and the physics of information. The prospect is that, with this invention, the individual self and its subjective consciousness may be maintained through neuromorphic emulation even after the biological brain has declined.

### BACKGROUND

A person's cognitive production is now sufficiently mediated by digital media (email, texts, documents, postings, photos, videos) that the generative capacity of an emulation can be tested for its identity fidelity—matching the person's essential identity—against this extensive and readily available behavioral output information with reasonable precision. Furthermore, evaluation and training of the Personal Neuromorphic Emulation (PNE) by the living person, such as in the form of a digital assistant helping with the tasks of daily life, provides the opportunity to further improve the fidelity of the emulation. The necessary tools for constructing the PNE according to the methods described in this invention are now available with the rapidly developing capability of generative artificial intelligence (AI).

In addition, high-resolution neuroimaging now provides extensive measurement of an individual's neurophysiology, including the electric fields of the cerebral cortex measured with high density electroencephalography (hdEEG), the hemodynamic function of the brain from functional magnetic resonance imaging (fMRI), and measures of brain function with fMRI and brain functional connectivity, and measures of brain structure using both fMRI and white matter tractography using diffusion tensor imaging.

There have been many recent inventions of neuromorphic, or brain-like, computational models (Shainline, et al., U.S. Pat. No. 11,258,415; Birdwell, et al., U.S. Pat. No. 10,929,745; Ritchey & Ritchey U.S. Pat. No. 11,287,847). However these are typically focused on more instrumental business or practical applications rather than the present goal of reconstruction of the personal mind in informatic (for example, digital) form.

Current Generative Pre-trained Transformers, such as GPT-4 and o1, are able to generate general-purpose (first-generation) emulations of human intelligence. Furthermore, there are neuromorphic neural network models such as the Virtual Brain (Jirsa, Sporns, Breakspear, Deco, & McIntosh, 2010; Sanz Leon et al., 2013), that can emulate the electrical fields of an individual cerebral cortex. However, these models have a limited range of functionality and are not sufficient to emulate the person's cognition, behavior, and subjective experience.

As starting points for building a detailed model of the person's neural connections, the connections of the human brain are given to a first approximation by the Human Connectome Project, as used in the generic brain network simulation used in this invention (Jiang, Gonzalez-Martinez, & Halgren, 2019). The physical connections are then specified further to emulate the individual's connectional architecture through diffusion tensor imaging and tractography reconstruction (Johansen-Berg et al., 2005) with the individual's brain (as now implemented in the inventors' Brain Electrophysiology Laboratory).

Finally, as described in the The Description of a Preferred Embodiment below, theoretical advances in information neuroscience have clarified that the entropy of life forms is not fundamentally different than that of matter and energy generally (Friston, 2019), so that brains, and minds, can be reconstructed fully through a sufficiently-resolved information representation.

### Objects and Advantages

The purpose of the present invention is to recreate the self (the human mind in its full cognitive function, personal memories, and subjective consciousness) in a durable informatic form. The practical advantage is to avoid deterioration of the self with brain aging and death, thereby achieving indefinite life extension.

The informatic definition of the self that is emerging from current scientific research and theory implies that the self is not restricted to its implementation in the biological body, but could be manifested and maintained with other (for example, digital) informatic forms.

At the same time, because the human mind is embodied (Johnson & Tucker, 2021), and currently limited to its neural implementation (Tucker & Luu, 2012), a neuromorphic emulation will be most comfortable if it reconstructs the full anatomical function of the human body, including its subcortical brain and visceral representations of the biological body (such as in the form of an emulation avatar).

Although an objective informatic formulation of the self is the key to this invention, to convincingly emulate the self requires a subjective validation as well. This requires a practical definition of the self that the person can understand and recognize to determine whether the personal neuromorphic emulation (PNE) is sufficiently accurate to be exchanged for the declining biological self. The several recent scientific advances reviewed in the Description of a Preferred Embodiment below have allowed an objective definition of the personal self that can now be implemented in informatic form in sufficient detail to be subjectively continuous with the historical self.

The invention draws from the ability to measure the neurophysiology of the brain, such as with high definition electroencephalography (hdEEG), such that the PNE can be trained to predict the brain's electrical fields in parallel with predicting the person's behavior. This is achieved through a high precision neuromorphic emulation, where the computational elements of the neural networks are not only arranged in an exact copy of the individual's neuroanatomy, but the neuronal-like elements have the electrical field generation properties of actual neurons. The result is reduced degrees of freedom of the emulation model: it must be functionally isomorphic to the person's brain and cannot simply predict their behavior with a generic neural network.

A second major advantage of the present invention is the extension of the training of the PNE to predict the person's joint brain activity and behavior not only during a large sample of waking behavior, but also during sleep and dreams over an extended interval of time (for example one year). The current evidence suggests that each day's experience is consolidated within the person's neural architecture through the specific mechanisms of the neurophysiology of sleep. During this consolidation, the neural patterns (connection weights) that define the knowledge of the self (the person's entire memory) are engaged and reflected in the electrical activity of the brain, including the knowledge of this historical self. Emulating these patterns of brain activity allow a reasonably short sample (for example, one year of recording) to capture the personal self in sufficient detail to construct a PNE that contains all the information of the person that is active at this stage of their life.

By achieving each of these objects, the PNE achieves the novel goal of replicating both the person's effective neural architecture and using/testing/refining this architecture to predict both the behavioral capacity (measured in the person's behavior) and the neurophysiological activity (captured in the brain's electrical fields and hemodynamic recordings during waking and sleep), with the same neural architecture and dynamic connection weights. This accuracy is then further validated (and refined where necessary) by both objective methods (predicting the person's behavior, such as from a digital corpus) and subjective methods (the subjective evaluation of the PNE's aesthetic choices).

### SUMMARY

Disclosed is a method for personal neuromorphic emulation. In a preferred embodiment the method may include constructing a computerized anatomical network model of neurons in the brain, the anatomical network model defining adjustably weighted connections between the modeled neurons and producing EEG signals as outputs; taking first hdEEG data from the subject while the subject's brain is awake and performing one or performing one or more first intellectual tasks; and first adjusting the weights of the weighted connections of the anatomical network model so as to drive the EEG signal outputs of the anatomical network model toward the first hdEEG data.

Also optionally, the method may include taking second hdEEG data from the subject while the subject's brain is undergoing REM sleep, taking third hdEEG data from the subject while the subject's brain is undergoing NREM sleep, taking fourth hdEEG data from the subject while the subject's brain is awake and performing one or more second intellectual tasks, and second adjusting the weights of the anatomical network model after the first adjusting so as to drive the EEG signal outputs of the anatomical network model toward all of the first, second, third, and fourth hdEEG data.

Also optionally, the method may include second adjusting the weights of the anatomical network model to fit the third hdEEG data, thereby emulating new learning of the subject's recent experiences, alternating with third adjusting the weights of the anatomical network model to fit the second hdEEG data, thereby emulating historical memory of the subject that is exercised and thus kept from catastrophic interference during REM sleep.

### BRIEF DESCRIPTION OF THE DRAWING

FIG. 1 shows the elements required for the construction, testing, and refining of the Personal Neuromorphic Emulation.

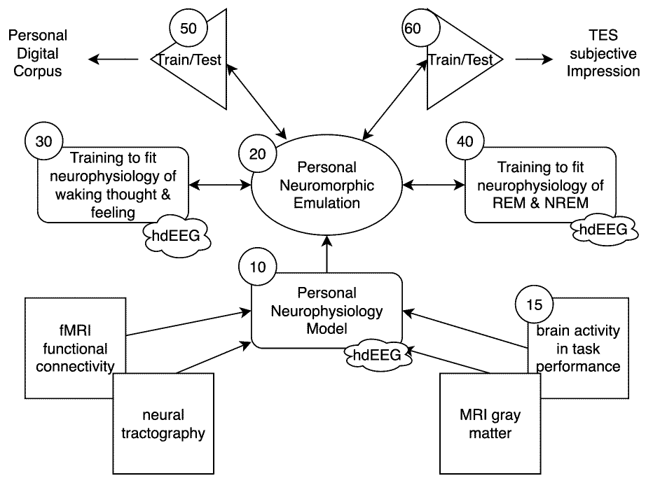

### DESCRIPTION OF A PREFERRED EMBODIMENT

The operation of this preferred embodiment depends on a clear understanding of the principles of operation on which it is based. These principles describe the information basis of physical intelligence, of which both biological and artificial intelligence are subsets. As described by Tucker & Luu (2024), the first principle is that the memory that allows concepts, now in machines as well as brains, is implemented in a distributed representation of connection weights among elementary processing neurons. The connection weights reflect associations, literally the strength of the associations among simple nodes or neurons in a large network architecture. For both brains and artificial intelligence, these networks are typically multi-leveled, so that the information is not simply at one associational level, but is achieved with multiple levels that can organize abstracted meaning at higher levels, thereby achieving abstract concepts. From this principle of distributed representational architecture, it is now clear that human brains and machines can operate on similar mechanisms of information processing.

Two additional principles are important to understanding the operation of the invention, one from neuropsychology and one from physics and information theory. For the second principle of physical intelligence, when we study how psychological function emerges from the activity of the human brain, we see that there is no separation of mind from brain. The mind is not just what the brain does, it is the brain (Changeux, 2002). More specifically, the functions of mind emerge from the neurodevelopmental process, the growth of the brain over time. This can be described as the principle of the neurodevelopmental identity of mind (Tucker & Luu, 2024). The mind, in its neural form, is constantly growing, constantly organizing its internal associations in exact identity with the strengthening or weakening of the brain's connections. Because the process of mind is always developing, the constituent neural connections of mind must also be dynamic, growing, and regulated in that growth. The neurodevelopmental process of mind is the physical substance of our psychological identities. Our psychological identities are then also (in exact register) profoundly developmental, changing and growing over time (Tucker & Luu, 2012). Importantly, this principle of neurodevelopmental identity, when combined with the third principle of informatic form of organisms (to be described next) will imply that the mind, and the process of subjective experience, can be reconstructed fully from an adequate neurodevelopmental process description.

There is an important corollary of the second principle, already well-known in machines and brains: in the dynamic growth of connections, plasticity is achieved at the expense of stability. The development of learning in distributed representations operates within the same generic connection/association space as existing knowledge, such that new learning invariably challenges the old memory. In the phenomenology of mind, careful reflection may teach us to recognize the old self that becomes at risk with each significant new learning experience (Tucker & Johnson, in preparation). As a result, the consolidation of memory in sleep involves one stage (NREM) that incorporates new information and another (REM) that reorganizes the self to accommodate to that new information without disrupting the prior information of the self (Tucker, Luu, & Friston, 2025).

The third principle of physical intelligence is important for understanding the fundamental nature of information, and why the information processing of mind is not restricted to its evolved biological form (brains). This is the principle of the informatic form of organisms. In physics we observe that the processes of matter, whether physical or chemical, tend toward greater entropy, meaning they tend to lose their complexity and release energy. Life has seemed to defy this fundamental rule of thermodynamics, maintaining its complexity through metabolism of external energy sources, homeostasis, and growth (Schrodinger, 1944).

Recent advances in the physics of information have provide a new perspective on the organization of information that defines a living organism as a non-equilibrium steady state (NESS), a self-organizing, growing form of information complexity that manifests rather defies the fundamental physics of entropy in the minimization of free energy (Friston, 2019). The cognitive functioning of this NESS—the self-evidencing that emerges in its operation as a good regulator (modeling its world for better or worse in order to live) can be fully specified in an information description that is not unique to minds, or brains, or even living things. Rather, the physics of information may offer an functional description of mind with the same terms that apply to physical, entropic, systems. The implication is that the neurodevelopmental process of organizing intelligence is not restricted to the particular biological form of evolved brains, but could be extended to a sufficiently complex informatic appliance of generic form.

Finally, an important practical point is that we can build tools to make tools. We do not need to build the PNE by hand, but instead could rely on a neuroscience-informed AI for the coding of a durable PNE. AI tools are now well-proven and rapidly-improving. We just need to train them to embody the essential neuromorphic principles.

### LIST OF REFERENCE NUMERALS

10 The Personal Neurophysiology Model is an initial computational replication of the person's brain in both structural and functional form. It is constructed from anatomical (fMRI functional connectivity, neural tractography, MRI gray matter) and behavioral (replicating brain activity in task performance) information from the person (15).
20 The Personal Neuromorphic Emulation is then the enduring replication of the individual brain-mind. It is first constructed from the Personal Neurophysiology Model, and is then trained (adjusting its neural connection weights) to fit each of the performance criteria (30 through 60) in order to achieve the functional equivalence of the PNE to the person's brain/mind.
30 This training element (neurophysiology of waking thought and feeling) exercises both the behavior and the neurophysiology of the PNE to be a high-fidelity replication of the individual mind, as measured through both physiology and behavior, in both objective (task performance, psychological assessment) and subjective (personal preference, subjective appraisal) domains.
40 This training element (training to fit neurophysiology of REM & NREM) uses the self-organization of the human brain during the stages of nightly sleep to optimize the characterization of the self.
50 A first validation test for the PNE (the Personal Digital Corpus) is its ability to reconstruct the person's behavior as captured in the personal digital corpus captured from the person's media and records.
60 A parallel validation test (TES subjective impression) draws upon the identity of neurophysiological with psychological structure. The PNE's interpretation of complex experiences (movies, literature, works of art) are compared with the person's. Alternatively, the PNE's prediction of the neural pattern of an experience is impressed on the brain through modulation of dynamic neural activity (which is isomorphic with consciousness). These methods perform the Subjective Turing Test: The PNE is found to be isomorphic with the self if it has the same feelings.

### DETAILED DESCRIPTION OF A PREFERRED EMBODIMENT

Construction of the PNE begins with the Personal Neurophysiology Model (10). The several structural and functional neuroimaging measures are derived for the person, at the optimum resolution available. A key design strategy is to recognize the difficulty of an exact biological replication, but the likely adequacy of a computational approximation. For example, the connectivity among the 100 billion or so neurons of a typical human brain is not decipherable from present methods, but, given current knowledge on the organization of cortical columns in each area of cerebral cortex, it is now feasible to reconstruct the individual's connectivity pattern among 200 million or so cortical columns, as may be suggested by relevant neuromorphic modeling (Csercsa et al., 2010).

Another form of evidence from structural imaging also emphasizes that the scale of modeling may not need to be at the molecular or even cellular level: the anatomy of subcortical systems is highly conserved across individuals (whereas cortical folding is highly idiosyncratic)(Tucker & Luu, 2012). As a result, generic subcortical models can be used as initiation sets for the individual's subcortical model and then trained in alternating fashion (such as in variational Bayes) with the training and construction of the individualized cerebral cortex. Thus, the individuality of the person's cortical activity requires corresponding individuality be impressed upon the weights generic subcortical model, so that the combined subcortical-cortical model accurately reflects the person's cortical electrical field (as localized with high density EEG) in waking and sleep as well as the person's behavior.

Although multiple brain measurement tools may be implemented to achieve the elements of this invention, the preferred embodiment uses high density electroencephalography (hdEEG; 10, 30, 40). This is because hdEEG has both temporal resolution and spatial resolution (using source localization with individual head conductivity), and because it is inexpensive and easily used for extensive recordings (such as extended recording during waking behavior and during many all-night sleep recordings over a year or even many years). The ideographic neural patterns then become highly characterized over extensive recording periods.

The Personal Neurophysiology Model (PNM)(10) is constructed to match the neural architecture of the person's brain, as specified initially by the structural neuroimaging technologies well known in the field and listed in the FIGURE as input to the PNM, and then as approximated (such as compute modules for the cortical columns in each region of cortex) in ways that are well known in the neuromorphic computation literature (and as explained in the Description of a Preferred Embodiment below). The approximations avoid reconstructing the cellular and molecular detail of the brain to simplify construction, subject to the constraint of computational adequacy in emulating the brain function.

The initial construction of the PNM begins with the individual's structural (such as T1-weighted) MRI. The method of tissue segmentation was described by one of the inventors in U.S. Pat. No. 8,478,011, and subsequently published in the scientific literature. [Li, K., Papademetris, X., & Tucker, D. M. 2016. BrainK for Structural Image Processing: Creating Electrical Models of the Human Head. Comput Intell Neurosci, 2016, 1349851.]. Once the cerebral cortex gray matter is segmented from other head tissues, the cortical surface is determined through the well-known marching cubes algorithm. In order to tessellate the surface of the cortex in variable resolution, a graph theory tessellation algorithm is used as detailed by Li and associates. In the preferred embodiment there are up to 9600 patches of equal size on the cortical surface (which is about 1200 sq cm in the human adult). This is implemented in the inventors' Sourcerer software, and an electrical source dipole is placed at the center of each patch. Although there are many cortical columns in each patch, the equivalent dipole is a first order representation if the cortical columns are mostly synchronous. With about 200 million (M) cortical columns in the human cortex, the 9600 patches would each represent about 20 thousand (K) columns, reflecting a reasonable first approximation for estimating regional cortical activity. By modeling each dipole patch (20K columns) with a generic computational model, adapted to the columnar architecture of that region of cortex (for example frontal pole versus primary somatosensory cortex) the training, if sufficiently extensive, can be expected to adapt the generic model for that cortical patch to a realistic emulation of the individual's cortical region.

The PNM includes the ability to compute both behavior (in the way that current AI networks compute behavior) and the electrophysiological fields of the cortex. Its connection weights will not be finely trained until it is transformed into the PNE (20), but they must both match the architecture of the human brain and allow computational adjustment on the basis of feedback from performance. The recent advances in active inference reviewed by Tucker & Luu (2021, 2023) provide the basis for this neuromorphic weight adjustment through feedback adjustment on predictive coding.

Essentially, the PNM is an accurate anatomical representation of the neural populations and connectivity of the person's individual brain, as described by the current Structural Model reviewed by Tucker & Luu 2023. Two approximations are made for the PNM in the preferred embodiment. The cortex is represented by computational neurons reflecting cortical columns as described above, and a generic subcortical model is used. This is because the human subcortex has much less idiosyncratic variability than the cerebral cortex. However, the generic connectivity of the subcortical model is adjustable, such that as the person's connection weights are more finely trained (after conversion to the PNE), the person's subcortical model is then adjusted (with the well-known variational Bayes approach of optimizing one component of the model while holding others constant) to be in synchrony to this operational tuning of the cortical model.

The PNM also generates electrical fields from each region of the cerebral cortex (reflecting the computational cortical columns or groups of columns). The inventor's current computational methods (in the Sourcerer software described at BEL.company) then estimates the activity of the 9600 patches, each of which is modeled by an electrical dipole that emulates the electrical field associated with activity (and thus connection weights) at that patch. Similar functional models for each cortical patch (or group of cortical columns) may be developed for hemodynamic activity, to allow training by the person's hemodynamic activity (fMRI or PET) during behavior and sleep, as is well known for The Virtual Brain (thevirtualbrain.org). A first-order functional adjustment of the PNM to assure both generation of behavior function and neurophysiological function can be done to match simple patterns of brain activity in task performance, as is well known from the extensive literature on hdEEG and fMRI/PET in human task performance.

Once the PNM (10) is completed and satisfactory, it is transformed into the PNE (20). Whereas the PNM (10) is a preliminary architecture, capturing both individual and generic human features but not required to function independently from its multiple software supports, the PNE (20) is a durable, self-organizing, standalone emulation, constructed to be trained for indefinite survival. The PNM is an initial template, and it can be effective in specifying the patterns to be emulated, even if specific functions are generated by separate software modules. The PNE (20) however, is a complete standalone neuromorphic computing system, prepared to be trained on an ongoing basis indefinitely. In the initial reduction to practice, the PNE may be constructed within a specialized computer for neural network computation comprising multiple GPUs (Graphics Processing Units, each of which is capable of reasonable computational implementation of a component of a neuromorphic architecture.

Thus the neural connections of the PNM (10) are a preliminary estimates for the person, but the durable self-organizing computational capacity of the PNE (20) allows its weights to be adjusted to be functionally accurate in predicting the evidence of the person's brain function in both waking cognition (30) and memory consolidation in sleep (40). At this point, there is an obvious isomorphism between the biological self and the PNE as it is exercised in the tasks and experiences of the day. There is an equally close, and now increasingly scientifically clarified, relation between the consolidation of memory during the stages of sleep and the maintenance of the self.

The operation of the PNE in emulating behavior, and being trained to do so adequately, follows the state of the art in machine learning (AI), subject to the constraint that the computations are achieved by the individual's approximate brain architecture, as exemplified by the inventors' scientific analysis of active inference (described in the Description of a Preferred Embodiment). Specifically, whereas weight corrections are achieved in many forms of AI by arbitrary methods such as back-propagation, the PNE must use neuromorphic methods such as described in the recent active inference literature.

Simultaneous to predicting behavior, the PNE predicts the individual's neurophysiological activity (exemplified for the preferred embodiment by the electrical fields of hdEEG) during waking task performance (30) and it predicts the individual's neurophysiology during sleep (40). Each night's sleep appears to reflect a new process of self-organizing the multiple developmental levels of the self. Emulation of this process over many nights (months, years) will provide the PNE with rich data on the ongoing (continually modified) developmental neural architecture of the self. With both the hdEEG during waking recording (30) and the sleep stage recording (40), the errors in testing this isomorphism (between the PNE and the waking and sleeping brain activity patterns) are fed back to improve the PNE. Multiple forms of neuromorphic learning may be used to improve this brain-machine emulation and thus the functional accuracy of the PNE. Although the constraint to fit both behavior and neurophysiology is a novel feature of the invention, the weight-fitting relies on the well-known (and rapidly developing) art of machine learning (AI).

Two additional domains of performance, the Personal Digital Corpus (the person's media records)(50) and the TES Subjective Impression (60) are used as validation measures, because they have clear face validity for determining the adequacy of the PNE. The person must decide when the PNE is an adequate representation of the self, to be trusted as the biological body fades. Although these are thus validation stages, there are likely to be errors in the PNE detected by this validation in the initial stages. Continued training can be then achieved during these validation tests, with the obvious requirement that validation must be repeated with new evidence that was not used in prior training.

The Personal Digital Corpus (50) may be continually increased during the extended training of the PNE, as needed, reflecting the continuing evolution of the functional self. The greater the resolution of the Personal Digital Corpus, the more effective is the training of the virtual self (20).

For the TES Subjective Impression (60), the evaluations of the PNE during a subjective task, such as evaluating art or other aesthetic experience, can be compared to the person's own subjective impressions. Alternatively, the electrical field of the PNE (measured with hdEEG) related to a psychological state (such as feeling anxious after a mistake, or such as smelling fresh cut grass) may be imposed upon the person's biological brain with methods such as high density Transcranial Electrical Stimulation (hdTES). The person's subjective response to this stimulation tests whether the electrophysiology is indeed identical with the experience. Again, error-correction is fed back to model training, and new validation must then be undertaken.

Taken together, these procedures provide a Subjective Turing Test, paralleling the objective Turing test performed by testing the PNE's emulation of the Personal Digital Corpus.

### BIBLIOGRAPHY

Changeux, J. P. 2002. The physiology of truth: Neuroscience and human knowledge: Harvard University Press.
Csercsa, R., Dombovari, B., Fabo, D., Wittner, L., Eross, L., Entz, L., . . . Ulbert, I. 2010. Laminar analysis of slow wave activity in humans. Brain, 133(9), 2814-2829.
Friston, K. 2019. A free energy principle for a particular physics. arXiv preprint arXiv: 1906.10184.
Jiang, X., Gonzalez-Martinez, J., & Halgren, E. 2019. Posterior hippocampal spindle ripples co-occur with neocortical theta bursts and downstates-upstates, and phase-lock with parietal spindles during NREM sleep in humans. Journal of Neuroscience, 39(45), 8949-8968.
Jirsa, V. K., Sporns, O., Breakspear, M., Deco, G., & McIntosh, A. R. 2010. Towards the virtual brain: network modeling of the intact and the damaged brain. Arch Ital Biol, 148(3), 189-205.
Johansen-Berg, H., Behrens, T. E., Sillery, E., Ciccarelli, O., Thompson, A. J., Smith, S. M., & Matthews, P. M. 2005. Functional-anatomical validation and individual variation of diffusion tractography-based segmentation of the human thalamus. Cereb Cortex, 15(1), 31-39.
Johnson, M., & Tucker, D. M. 2021. Out of the Cave: The Natural Philosophy of Mind and Knowing. Cambridge, M A: MIT Press.
Sanz Leon, P., Knock, S. A., Woodman, M. M., Domide, L., Mersmann, J., McIntosh, A. R., & Jirsa, V. 2013. The Virtual Brain: a simulator of primate brain network dynamics. Front Neuroinform, 7, 10.
Schrodinger, E. 1944, What is Life? The Physical Aspect of the Living Cell. Cambridge, England: Cambridge University Press.
Tucker, D. M., & Johnson, M. in preparation, Deep Feelings: The Intuitive Base of Experience.
Tucker, D. M., & Luu, P. 2012. Cognition and Neural Development. New York: Oxford University Press.
Tucker, D. M., & Luu, P. 2024. Feasibility of a Personal Neuromorphic Emulation. Entropy, 26, 759.
Tucker, D. M., Luu, P., & Friston, K. J. 2025. Consolidating Active Inference in Sleep.

1. A method for personal neuromorphic emulation of a subject's brain, comprising:
constructing a computerized anatomical network model of neurons in the brain, the anatomical network model defining adjustably weighted connections between the modeled neurons and producing EEG signals as outputs;
taking first hdEEG data from the subject while the subject's brain is awake and performing one or more first intellectual tasks; and
first adjusting the weights of the weighted connections of the anatomical network model so as to drive the EEG signal outputs of the anatomical network model toward the first hdEEG data.

2. The method of claim 1, further comprising taking second hdEEG data from the subject while the subject's brain is undergoing REM sleep, taking third hdEEG data from the subject while the subject's brain is undergoing NREM sleep, taking fourth hdEEG data from the subject while the subject's brain is awake and performing one or more second intellectual tasks, and second adjusting the weights of the anatomical network model after said first adjusting so as to drive the EEG signal outputs of the anatomical network model toward all of the first, second, third, and fourth hdEEG data.

3. The method of claim 2, further comprising third adjusting the weights of the anatomical network model to fit the third hdEEG data, thereby emulating new learning of the subject's recent experiences, alternating with fourth adjusting the weights of the anatomical network model to fit the second hdEEG data, thereby emulating historical memory of the subject that is exercised and thus kept from catastrophic interference during REM sleep.

4. The method of claim 1, further comprising second adjusting the weights of the anatomical network model to fit the third hdEEG data, thereby emulating new learning of the subject's recent experiences, alternating with fitting the weights to the second hdEEG data, thereby emulating historical memory of the subject that is exercised and thus kept from catastrophic interference during REM sleep.
